(19) United States
(12) Patent Application Publication
(10) Pub. No.: US 2026/0212055 A1
(43) Pub. Date: Jul. 23, 2026

(54) INFORMATION ANALYSIS METHOD AND INFORMATION ANALYZER

(71) Applicant: NTT, Inc., Tokyo (JP)

(72) Inventors: Kazuhiro TAKAYA, Musashino-shi (JP); Sosuke IMAMURA, Musashino-shi (JP)

(73) Assignee: NTT, Inc., Tokyo (JP)

(21) Appl. No.: 18/714,249

(22) PCT Filed: Dec. 9, 2021
(86) PCT No.: PCT/JP2021/045380

§ 371 Date: May 29, 2024

(51) Int. Cl. G06F 30/10; C12N 15/82; G16B 40/00

(52) U.S. Cl. CPC G06F 30/10; C12N 15/8201; G16B 40/00

(57) ABSTRACT

An information analysis method includes: setting a growing environment of a plant to be subjected to genome editing in an environmentally controllable closed space; preparing a plurality of kinds of variants in which one gene of the plant is genome-edited, and growing the plurality of kinds of variants simultaneously in the closed space; creating time series data in which first data indicating a growing environment of the plurality of kinds of variants and second data indicating a growth state of the plurality of kinds of variants are recorded in time synchronization; and creating an analysis model representing a correlation among gene information, a growing environment, and a growth state using gene information regarding the plurality of kinds of variants and the time series data.

### TECHNICAL FIELD

Embodiments relate to an information analysis method and an information analysis device.

### BACKGROUND ART

Efforts have been made to find an optimal growing environment by monitoring growth data of variants and F1 varieties created by gene recombination or genome editing. In addition, studies have been conducted to find a relationship between past growing data and cultivation conditions and estimate growing results.

### CITATION LIST

### Patent Literature

Patent Literature 1: JP 2004-121093 A
Patent Literature 2: JP 2021-045063 A
Patent Literature 3: JP 2017-051118 A

### Non Patent Literature

Non Patent Literature 1: Machiko Fukuda, “The expression level of a leaf FT-like gene of leaf lettuce increases with the flower bud development at the stem tip”, [online]National Agriculture and Food Research Organization, [Searched on Nov. 30, 2021], Internet <URL:https://www.naro.go.jp/project/results/laboratory/vege tea/2011/113a4_10_04.html>

### SUMMARY OF INVENTION

### Technical Problem

However, there have been problems that optimization and improvement of a cultivation process are based on skills and experiences of researchers, it is difficult to apply to a case where a change in a trait is nonlinear with respect to environmental factors, and it is not possible to calculate optimum environmental conditions from a gene expression level.

Therefore, an object of the present invention is to establish a method for evaluating an effect of genome editing by fusing “research results related to breeding and raising seedling by a plant factory” and “information communication technology (data collection, data analysis, numerical simulation, and the like)”.

### Solution to Problem

An information analysis method according to an embodiment includes: setting a growing environment of a plant to be subjected to genome editing in an environmentally controllable closed space; preparing a plurality of kinds of variants in which one gene of the plant is genome-edited, and growing the plurality of kinds of variants simultaneously in the closed space; creating time series data in which first data indicating a growing environment of the plurality of kinds of variants and second data indicating a growth state of the plurality of kinds of variants are recorded in time synchronization; and creating an analysis model representing a correlation among gene information, a growing environment, and a growth state using gene information regarding the plurality of kinds of Variants and the time series data.

### Advantageous Effects of Invention

According to the information analysis method according to the embodiment, it is possible to improve productivity of excellent variety plants and create vegetation having desired environmental adaptability.

### BRIEF DESCRIPTION OF DRAWINGS

FIG. 1 is a block diagram illustrating one example of a configuration of an information analysis system according to a first embodiment.

FIG. 2 is a block diagram illustrating an example of a configuration of an information analysis device according to the first embodiment.

FIG. 3 is a conceptual diagram illustrating an example of a configuration of an artificial plant raising site according to the first embodiment.

FIG. 4 is a block diagram illustrating an example of a functional configuration of the information analysis device according to the first embodiment.

FIG. 5 is a table illustrating an example of a data format of time series data in the first embodiment.

FIG. 6 is a flowchart illustrating an example of analysis processing using the information analysis system according to the first embodiment.

FIG. 7 is a conceptual diagram of an optimization problem related to early prediction confirmation of genome editing accuracy using an analysis model generated by the analysis processing according to the first embodiment.

FIG. 8 is a conceptual diagram illustrating an example of growth simulation by the information analysis device according to the first embodiment.

FIG. 9 is a conceptual diagram illustrating an example of a configuration of an artificial plant raising site according to a second embodiment.

FIG. 10 is a flowchart illustrating an example of analysis processing using an information analysis system according to the second embodiment.

FIG. 11 is a block diagram illustrating an example of a configuration of an artificial plant raising site according to a third embodiment.

### DESCRIPTION OF EMBODIMENTS

Hereinafter, embodiments will be described with reference to the drawings. The embodiments illustrate a device and a method for embodying the technical idea of the invention. The drawings are schematic or conceptual. In the present specification, the same reference numerals are added to components that have substantially the same functions and configurations. Numbers and the like added to reference numerals are referred to by the same reference numerals and used to distinguish between similar elements.

### <1> First Embodiment

A first embodiment relates to a method for predicting a growing result of a genome-edited plant (that is, a variant) in a case where a certain growing environment is prepared, or a change in a trait to be an effect of genome editing. Hereinafter, an information analysis system 1 according to the first embodiment will be described.

### <1-1> Configuration

### <1-1-1> Configuration of Information Analysis System 1

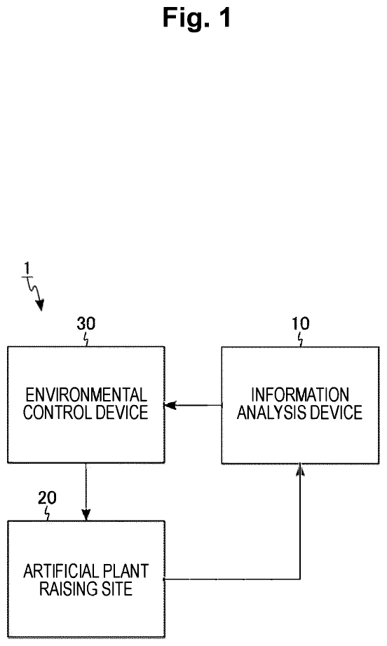

FIG. 1 is a block diagram illustrating an example of a configuration of the information analysis system 1 according to the first embodiment. As illustrated in FIG. 1, the information analysis system 1 includes, for example, an information analysis device 10, an artificial plant raising site 20, and an environmental control device 30.

The information analysis device 10 is a computer capable of creating an analysis model related to genome editing of a plant by analyzing and learning input data. The information analysis device 10 is configured to be able to communicate with each of the artificial plant raising site 20 and the environmental control device 30. Wireless communication or wired communication may be used for a network used for communication of the information analysis device 10, the artificial plant raising site 20, and the environmental control device 30. The “analysis model” may be referred to as a “learning model” or a “growth model”.

The artificial plant raising site 20 is a closed space used for growing a plant. The artificial plant raising site 20 is configured to be able to artificially control an environment (that is, a growing environment of a plant) by the control of the environmental control device 30. The artificial plant raising site 20 can transmit data regarding a growing situation and a growing environment to the information analysis device 10. The artificial plant raising site 20 may be referred to as a “plant factory”, a “closed plant cultivation space”, a “completely closed plant factory”, or a “completely closed plant factory”.

The environmental control device 30 is a computer that comprehensively controls a plurality of control devices for controlling an environment of the artificial plant raising site 20. The environmental control device 30 controls the plurality of control devices on the basis of control of the information analysis device 10, and forms a desired growing environment in the artificial plant raising site 20. The environmental control device 30 can control, for example, a light amount, air temperature, water temperature, a water supply amount, carbon dioxide concentration, nutrients, and the like in the artificial plant raising site 20. Note that the environmental control device 30 may be provided in the artificial plant raising site 20.

### <1-1-2> Configuration of Information Analysis Device 10

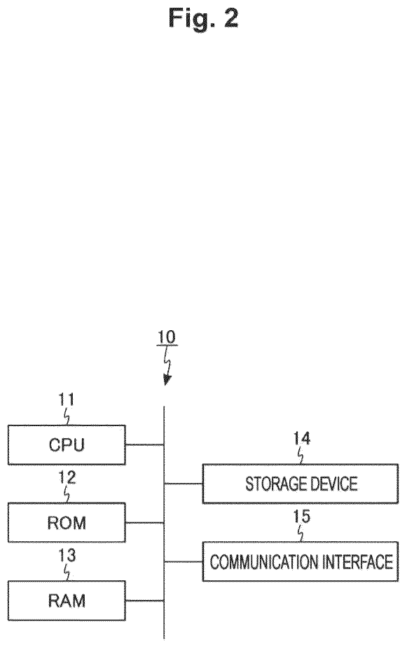

FIG. 2 is a block diagram illustrating an example of a configuration of the information analysis device 10 according to the first embodiment. As illustrated in Fig. 2, the information analysis device 10 includes, for example, a central processing unit (CPU) 11, a read only memory (ROM) 12, a random access memory (RAM) 13, a storage device 14, and a communication interface 15.

The CPU 11 is an integrated circuit capable of executing various programs. The CPU 11 controls an entire operation of the information analysis device 10.

The ROM 12 is a nonvolatile semiconductor memory. The ROM 12 stores programs, control data, and the like for controlling the information analysis device 10.

The RAM 13 is, for example, a volatile semiconductor memory. The RAM 13 is used as a working area of the CPU memory. 11.

The storage device 14 is a nonvolatile storage device. The storage device 14 stores, for example, system software of the information analysis device 10, data acquired via a network, and the like.

The communication interface 15 is a communication circuit configured to be connectable to a network. The information analysis device 10 can transfer data (information) received via the communication interface 15 to the RAM 13 or the storage device 14, and output an analysis result of the data to an external device via the communication interface 15.

The information analysis device 10 can realize a functional configuration described later by executing a program. Note that a hardware configuration of the information analysis device 10 may be another configuration. A display, an input interface, a detachable storage device, and the like may be connected to the information analysis device 10. The information analysis device 10 may display a simulation result or the like on the display.

### <1-1-3> Configuration of Artificial Plant Raising Site 20

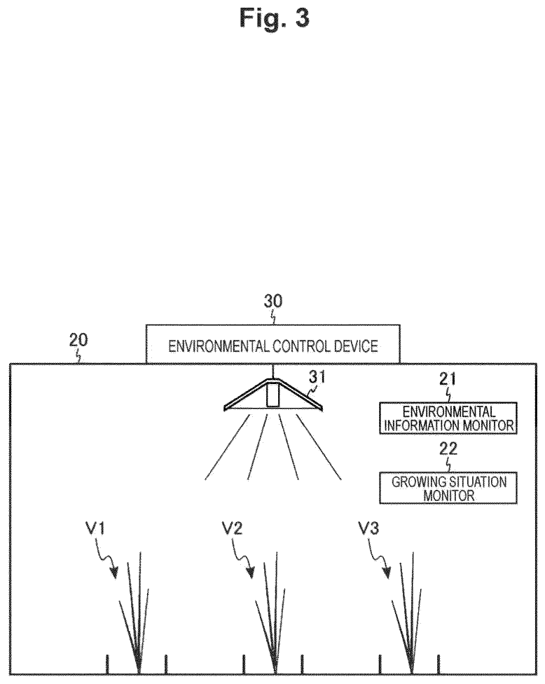

FIG. 3 is a conceptual diagram illustrating an example of a configuration of the artificial plant raising site 20 according to the first embodiment. As illustrated in Fig. 3, the artificial plant raising site 20 cultivates a plurality of kinds of plants (variants) obtained by genome editing different genes (genome-edited strains) in the same growing environment. Specifically, the artificial plant raising site 20 cultivates a plurality of kinds of variants V1, V2, and V3 which are of a plant to be subjected to genome editing and, for example, obtained by genome editing one different gene. The plurality of kinds of variants V1, V2 and V3 is arranged in a distinguishable manner in the artificial plant raising site 20. Note that the number of kinds of variants in which the artificial plant raising site 20 grows is only required to be plural and is not limited to three.

In addition, the artificial plant raising site 20 includes, for example, an environmental information monitor 21, a growing situation monitor 22, and a lighting device 31. Each of the environmental information monitor 21 and the growing situation monitor 22 is connected to the information analysis device 10. The lighting device 31 is connected to the environmental control device 30.

The environmental information monitor 21 is a device that monitors environmental conditions in the artificial plant raising site 20. The environmental information monitor 21 includes, for example, a light amount sensor, an air temperature sensor, a water temperature sensor, a water supply amount sensor, a humidity sensor, a carbon dioxide (CO₂) concentration sensor, and a nutrient sensor. The environmental information monitor 21 transmits environmental information (environmental parameters or variables) of the artificial plant raising site 20 detected by various sensors to the information analysis device 10 as environmental information data 210. That is, the environmental information data 210 includes any one of information of a light amount, air temperature, water temperature, a water supply amount, carbon dioxide concentration, nutrients, and time.

The growing situation monitor 22 is a device that monitors a growth process of each of the plurality of kinds of variants V1, V2, and V3 in the artificial plant raising site 20. The growing situation monitor 22 includes, for example, a camera and an analysis device. The growing situation monitor 22 has a function of analyzing an image acquired by the camera by the analysis device. Note that the environmental information monitor 21 and the growing situation monitor 22 may share a sensor or the like. The growing situation monitor 22 can analyze a total photosynthesis rate, a dark respiration rate, chlorophyll fluorescence, pore conductance, a leaf area/leaf number distribution, a leaf inclination angle distribution, a root structure and distribution, a chemical component distribution, a net photosynthesis rate, a transpiration rate, a respiration rate, a carbon dioxide application rate, a water supply rate, a water absorption rate, electric power, and the like as information indicating a growth degree of each variant. Then, the growing situation monitor 22 transmits information indicating a growing situation of each of the plurality of kinds of variants to the information analysis device 10 as growing information data 220. That is, the growing information data 220 includes any information of the total photosynthesis rate, the dark respiration rate, the chlorophyll fluorescence, the pore conductance, the leaf area/leaf number distribution, the leaf inclination angle distribution, the root structure and distribution, the chemical component distribution, the net photosynthesis rate, the transpiration rate, the respiration rate, the carbon dioxide application rate, the water supply rate, the water absorption rate, and the electric power.

The lighting device 31 is a lighting installed in the artificial plant raising site 20. The lighting device 31 irradiates each of the plurality of kinds of variants V1, V2, and V3 with light. An amount of light emitted by the lighting device 31 and the like can be controlled by the environmental control device 30. Note that the artificial plant raising site 20 may include other control devices. Examples of the other control devices will be described in a third embodiment.

### <1-1-4> Functional Configuration of Information Analysis Device 10

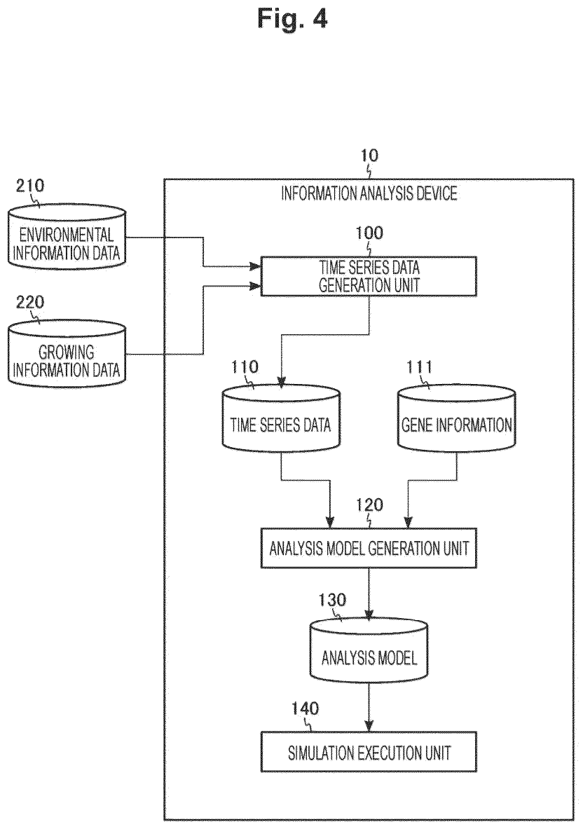

FIG. 4 is a block diagram illustrating an example of a functional configuration of the information analysis device 10 according to the first embodiment. As illustrated in FIG. 4, the information analysis device 10 includes, for example, a time series data generation unit 100, time series data 110, gene information 111, an analysis model generation unit 120, an analysis model 130, and a simulation execution unit 140.

The time series data generation unit 100 records by synchronizing recording times of the environmental information data 210 input from the environmental information monitor 21 and the growing information data 220 input from the growing situation monitor 22 to generate the time series data 110. In other words, the time series data generation unit 100 synchronizes the environmental information (environmental parameters and variables) with the growth process of the cultivated and grown plant, and accumulates the synchronized information in the time series data 110. The time series data 110 is used as teacher data for creating the analysis model 130 of growth simulation.

The analysis model generation unit 120 generates or updates the analysis model 130 by learning using the time series data 110 and the gene information 111. The gene information 111 includes information on genes before and after genome editing of the variant species grown by the artificial plant raising site 20. The analysis model 130 is a model for evaluating a growth process of a variant from gene information and environmental information. The simulation execution unit 140 executes a growth simulation of a plant (variant) on the basis of the analysis model 130. In the growth simulation, when gene information of a variant and growing environmental information are input, the simulation execution unit 140 simulates a growth process of the variant based on the analysis model 130.

### <1-1-5> Data Format of Time Series Data 110

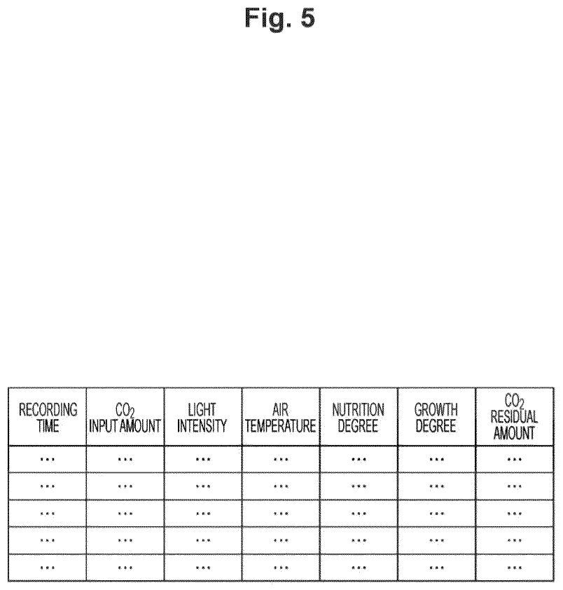

FIG. 5 is a table illustrating an example of a data format of the time series data 110 in the first embodiment. As illustrated in FIG. 5, the time series data 110 records, for example, time series data of recording time, a CO₂ input amount, light intensity (light amount), air temperature, a nutrition degree, a growth degree, and a CO₂ residual amount. In this example, the artificial plant raising site 20 measures the residual amount of carbon dioxide (CO₂ residual amount) in the artificial plant raising site 20 using carbon dioxide as an input. Then, the information analysis device 10 records the environmental information (environmental information data 210) and the growth process (growing information data 220) as the time series data 110. The artificial plant raising site 20 may measure the amount of carbon dioxide absorbed by the variants and record the amount in the time series data 110. In addition, the time series data 110 may be recorded together with the gene information 111.

### <1-2> Analysis Processing

Next, analysis processing using the information analysis system 1 according to the first embodiment will be described. The information analysis device 10 can execute analysis processing for each cultivation cycle. In the present specification, the “cultivation cycle” corresponds to a cycle in which seeds or seedlings of a plant (variant) are grown in the artificial plant raising site 20 and the growing of the plant is completed (for example, harvested or discarded). Specifically, “one cycle of the cultivation cycle” corresponds to a series of processing including the growing of the genome-edited plant (that is, variant) and the creation of the analysis model 130 based on a growing result.

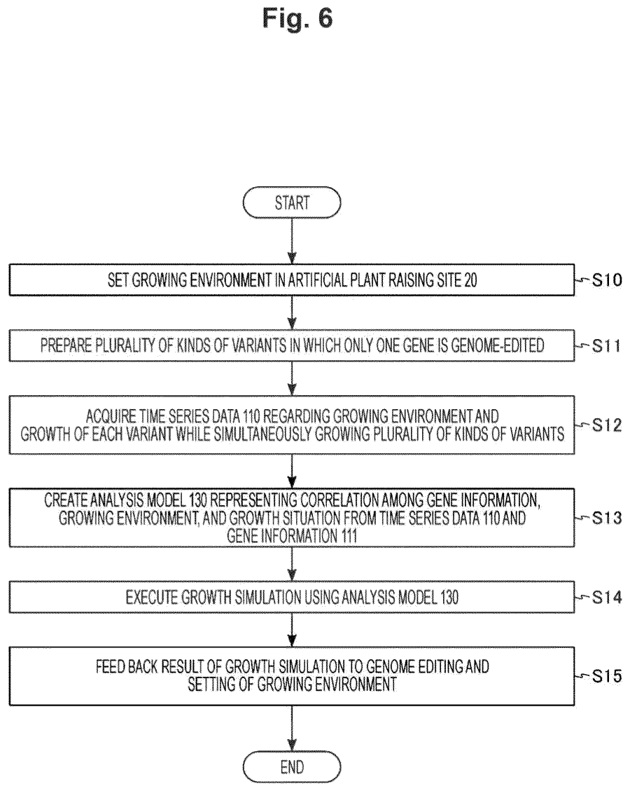

FIG. 6 is a flowchart illustrating an example of analysis processing using the information analysis system 1 according to the first embodiment. FIG. 6 illustrates processing executed for each cultivation cycle. Hereinafter, analysis processing of the information analysis system 1 according to the first embodiment will be. described with reference to FIG. 6.

First, a growing environment is set in the artificial plant raising site 20 (S10). As the setting of the growing environment, for example, intensity of a light amount of the lighting device 31 is set as a first condition, a level of temperature in the artificial plant raising site 20 1s set as a second condition, and the rich and poor of fertilizer to be given to each variant in the artificial plant raising site 20 is set as a third condition.

Next, a plurality of kinds of variants in which only one gene is genome-edited is prepared (S11). A type of gene to be subjected to genome editing can be freely selected. The type of gene to be subjected to genome editing may be selected on the basis of a result of past cultivation cycle analysis processing. The plurality of kinds of variants is distinguishably installed in the artificial plant raising site 20. For example, seeds and seedlings of the plurality of kinds of variants are respectively arranged in a plurality of regions in the artificial plant raising site 20,

Next, while simultaneously growing the plurality of kinds of variants, the time series data 110 regarding the growing environment and the growth of each variant is acquired (S12). Specifically, the environmental control device 30 controls each control device in the artificial plant raising site 20 so as to realize the growing environment on the basis of the conditions of the growing environment set in S10. More specifically, in a closed plant cultivating/growing space, the environmental control device 30 controls growing environmental conditions such as water, carbon dioxide, light, and nutrients, and supplies the target variants to grow each variant. At this time, the environmental information monitor 21 and the growing situation monitor 22 monitor a growth degree, a respiration amount, and the like of each variant in a growing process. Then, the generated environmental information data 210 and growing information data 220 are synchronized and accumulated in the information analysis device 10 as the time series data 110. In other words, in the growing process of the plurality of kinds of variants, the environmental information monitor 21 and the growing situation monitor 22 respectively transmit the environmental information data 210 and the growing information data 220 to the information analysis device 10, and the time series data generation unit 100 of the information analysis device 10 generates the time series data 110 using the received environmental information data 210 and growing information data 220.

Next, from the time series data 110 and the gene information 111, the analysis model 130 representing a correlation among gene information, a growing environment, and a growth situation is created (S13). Specifically, the analysis model generation unit 120 performs machine learning on a correlation between the growing and the growth of each variant from the time series data 110 and the gene information 111 to create the analysis model 130.

Next, a growth simulation is executed using the analysis model 130 (S14), In the growth simulation, the simulation execution unit 140 evaluates a difference in growth degree or the like between different variants of a genome-edited strain.

Next, a result of the growth simulation is fed back to genome editing and setting of a growing environment (S15). When processing of S15 is completed, the information analysis device 10 ends the analysis process corresponding to one cultivation cycle.

The information analysis device 10 can update the analysis model 130 on the basis of the new time series data 110 and gene information 111 obtained by changing the setting of the growing environment for each cultivation cycle and collecting the environmental information data 210 and the growing information data 220. In the next cultivation cycle in which feedback has been received from the result of the one-cycle analysis processing described above, it is conceivable to perform cultivation of another edited strain series having different functions and the like in the same growing environment, or to perform cultivation in which the growing environment is changed using the same genome-edited strain series. The analysis model 130 updated by repeating the analysis processing including the feedback can be used for searching for a combination of an optimal genome-edited gene (genome-edited strain) and a growing environment that maximizes the growth degree.

Note that the gene information of the plant obtained in one cultivation cycle, the conditions of the growing environment to be set, and the measurement results of the plant growth process are finite. Therefore, in a case where the machine learning is executed using only the actual measurement result, an error can occur due to a shortage of the amount of data used for learning by the analysis model generation unit 120 or a variation in data. Therefore, the information analysis device 10 may use logistic function approximation, autoregressive regression, multiple regression, or the like to perform formulation of a causal relationship from the cultivation data and correction of the teacher data using the causal relationship formula. As a result, the information analysis device 10 can improve accuracy of the growth simulation.

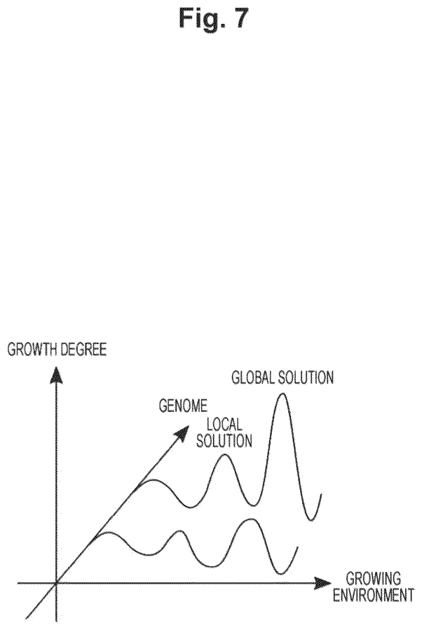

FIG. 7 is a conceptual diagram of an optimization problem related to early prediction confirmation of genome editing accuracy using the analysis model 130 generated by the analysis processing according to the first embodiment. In the graph shown in FIG. 7, the X axis corresponds to the growing environment, the Y axis corresponds to the genome, and the Z axis corresponds to the growth degree. In FIG. 7, the relationship among the growing environment, the genome, and the growth degree is three-dimensionally expressed, but actually, each of the growing environment axis, the genome axis, and the growth degree axis is multidimensional.

This example shows a problem of experimentally finding an optimal solution with the growth degree as an evaluation function and each of the genome axis and the growing environment axis as a variable. By feeding back a result of such a growth simulation to genome editing and setting of a growing environment, a genome editing parameter and a growing environment parameter can be automatically improved. In order to speed up convergence of a prediction result in a process of finding a global optimum solution (global solution) that maximizes the growth degree rather than a local solution with a locally high growth degree in a plane constituted by variations of the growing environment and variations of the genome editing, it is preferable to use machine learning or numerical simulation.

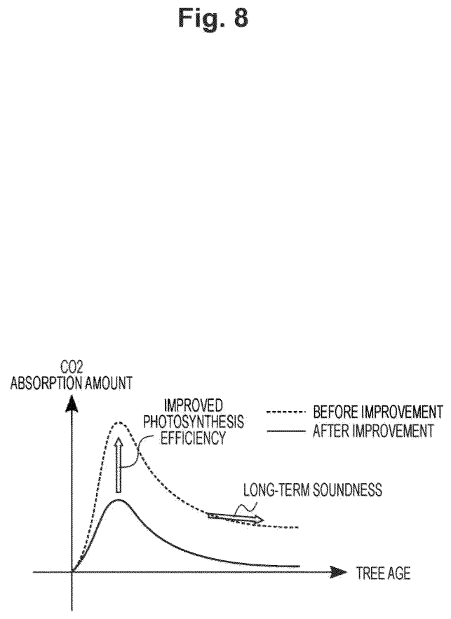

FIG. 8 is a conceptual diagram illustrating an example of growth simulation by the information analysis device 10 according to the first embodiment. FIG. 8 shows a relationship between a tree age and a CO₂ absorption amount (tree age dependency of a carbon dioxide absorption amount) before and after improvement of the plant. As shown in FIG. 8, under a certain growing environment, photosynthesis efficiency of the plant after the improvement is improved as compared with that before the improvement. Then, it is estimated that the improved plant exhibits long-term soundness and longer life as a trait change than those before the improvement. The growth simulation can also predict a genome editing target necessary for such a trait change and a gene sequence after genome editing. As a result, a user can reproduce and verify optimal gene information and growing environmental conditions based on a prediction result by the growth simulation in real space.

Here, a method for selecting an excellent variety by retroactive evaluation (that is, evaluation in a time domain) of a plant growth process will be described. First, a growth process (growing information data 220) and a cultivation process (environmental information data 210) including an environmental setting are recorded for the selected seeds or seedlings. Then, the information analysis device 10 learns a correlation between environment and growth. This makes it possible to select excellent seeds or seedlings suitable for obtaining a desired trait. Furthermore, the information analysis device 10 records the growth process of the selected seeds or seedlings as the time series data 110, so that it is possible to analyze an environmental condition, a cultivation condition, or the like in which a good trait change or a trait change different from a desired trait change appears. For example, by comparing growth processes of different seeds or seedlings under the same environmental conditions and cultivation conditions, the information analysis device 10 can accurately compare at which point a superior difference appears.

### <1-3> Advantageous Effects of First Embodiment

The information analysis system 1 according to the first embodiment includes a closed space (artificial plant raising site 20) capable of artificially realizing various growing environments without being affected by environmental changes outside the space. Then, the information analysis device 10 synchronizes the environmental data when the environmental conditions in the closed space are changed with the plant growing data and collects the data as the time series data 110, thereby creating the analysis model 130 capable of simulating the growth process in any growing environment from the correlation between the environment and the growth.

As a result, the simulation execution unit 140 can perform a virtual growth simulation in which any growing period is selected using the analysis model 130, and can estimate a growth process of the plant with respect to the environmental conditions. At the same time, by comparing growth processes before and after genome editing, effects of genome editing and the like can be evaluated. Furthermore, the analysis model generation unit 120 creates the analysis model 130 by machine learning of the correlation between the environmental conditions for a plurality of kinds of variants differing only in one gene and the growing and growth of each variant, whereby the simulation execution unit 140 can estimate the growth process of the plant with respect to the environmental conditions for the genetic characteristics of each variant. Then, by feeding back the simulation result to the cultivation method and the environmental setting in the actual cultivation space, the optimum environmental conditions for the growth of plants having certain genetic characteristics can be efficiently searched with high accuracy.

In addition, in the information analysis system 1 according to the first embodiment, a plurality of kinds of variants in which only one gene is genome-edited (genes to be edited are different) is prepared for a target plant to be grown/cultivated, the plurality of kinds of variants is grown in the same environmental conditions in the closed space, and growing data and environmental data of each variant are acquired in a set.

As a result, the information analysis device 10 can create the analysis model 130 representing the correlation with the gene information in addition to the correlation between the environment and the growth. In addition, the information analysis device 10 can perform a growth simulation for any growth period using the analysis model 130 and feed back a simulation result to a cultivation method and environmental setting in the actual cultivation space. Furthermore, the information analysis device 10 can predict variation of the plant under a certain growing environment in a case where genetic modification equivalent to the variation when the plant adapts to a change in a certain environment is performed. As a result, the information analysis device 10 can efficiently and accurately search for genetic characteristics optimal for a certain growing environment and target selection of genome editing necessary for obtaining the genetic characteristics.

As described above, the information analysis system 1 according to the first embodiment (1) can construct a desired growing environment in a closed space and control environmental parameters, (2) can establish a method for evaluating whether a change in a trait is caused by an effect of genome editing or whether a plant or the like is adapted to an environment, (3) can perform numerical modeling of plant growing information and environmental information (quality, a growth degree, a CO₂ absorption amount, etc.), (4) can establish a method for acquiring plant growth data in a time domain (the time series data 110 synchronized with the environmental parameters), (5) can perform selection of an excellent variety by virtual growth simulation, search of an ideal growing environment, and selection of an optimum variety under a certain growing environment, and (6) can perform growth simulation reflecting a genome editing method, and feed back control of a growing environment in which a desired growth process can be expected to be automatically improved. Therefore, the information analysis system 1 according to the first embodiment can improve productivity of excellent variety plants and create vegetation having desired environmental adaptability.

### <2> Second Embodiment

A second embodiment relates to an information analysis system 1 that executes analysis processing similar to that of the first embodiment by using an artificial plant raising site 20A having a plurality of growing environmental cells. Hereinafter, the information analysis system 1 according to the second embodiment will be described in terms of differences from the first embodiment.

### <2-1> Configuration of Artificial Plant Raising Site 20A

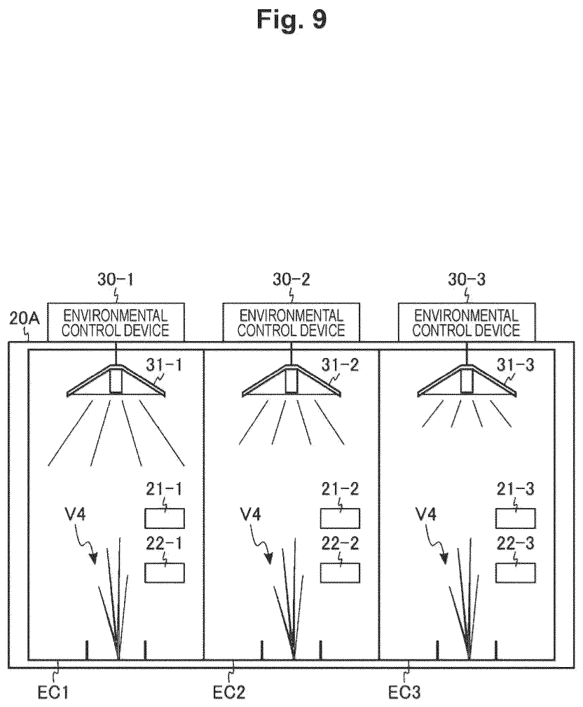

FIG. 9 is a conceptual diagram illustrating an example of a configuration of an artificial plant raising site 20A according to the second embodiment. As Illustrated in Fig. 9, the artificial plant raising site 20A cultivates the same variant (that is, a plant having the same genome- edited gene and strain) in a plurality of growing environments. Specifically, the artificial plant raising site 20A includes a plurality of growing environmental cells EC1, BC2, and EC3. Each of the plurality of growing environmental cells EC1, EC2, and RC3 is an independently provided closed space. In the artificial plant raising site 20A, an environmental control device 30, an environmental information monitor 21, a growing situation monitor 22, and a lighting device 31 are provided for each of the plurality of growing environmental cells EC.

Specifically, the growing environmental cell EC1 has a growing environment controlled by an environmental control device 30-1, and includes an environmental information monitor 21-1, a growing situation monitor 22-1, and a lighting device 31-1. The growing environmental cell EC2 has a growing environment controlled by an environmental control device 30-2, and includes an environmental information monitor 21-2, a growing situation monitor 22-2, and a lighting device 31-2. The growing environmental cell EC3 has a growing environment controlled by an environmental control device 30-3, and includes an environmental information monitor 21-3, a growing situation monitor 22-3, and a lighting device 31-3. Each of the growing environmental cells EC1, EC2, and EC3 grows, for example, a variant V4.

Each environmental control device 30 can operate independently on the basis of an instruction from an information analysis device 10. The environmental information monitors 21-1, 21-2, and 21-3 monitor environmental conditions in the growing environmental cells EC1, EC2, and EC3, respectively. The growing situation monitors 22-1, 22-2, and 22-3 monitor a growth process of the variant V4 in the growing environmental cells EC1, EC2, and EC3, respectively. Each environmental information monitor 21 transmits a monitoring result to the information analysis device 10 as environmental information data 210. Each growing situation monitor 22 transmits a monitoring result to the information analysis device 10 as growing information data 220. The lighting devices 31-1, 31-2, and 31-3 irradiate the variant V4 in the growing environmental cells EC1, EC2, and EC3 with light, respectively.

Note that the number of sets of the growing environmental cell EC, the environmental control device 30, the environmental information monitor 21, the growing situation monitor 22, and the lighting device 31 included in the artificial plant raising site 20A is only required to be plural and is not limited to three. The artificial plant raising site 20 of the first embodiment may be used in the information analysis system 1 according to the second embodiment. In this case, each of the plurality of artificial plant raising sites 20 grows the same variant. Then, the information analysis device 10 sets different growing environments for each artificial plant raising site 20. Other configurations of the information analysis system 1 according to the second embodiment are similar to those of the first embodiment.

### <2-2> Analysis Processing

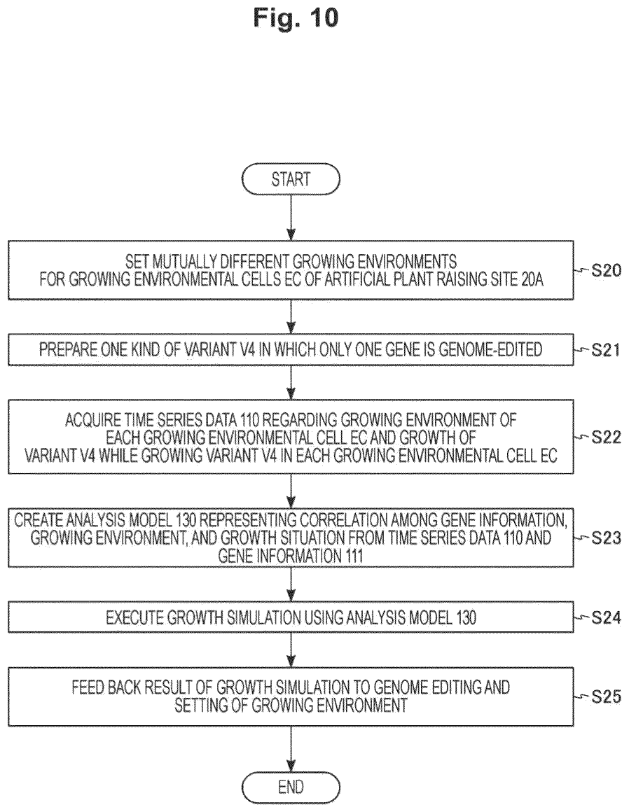

FIG. 10 is a flowchart illustrating an example of analysis processing using the information analysis system 1 according to the second embodiment. Hereinafter, analysis processing of the information analysis system 1 according to the second embodiment will be described with reference to FIG. 10.

First, mutually different growing environments are set for the growing environmental cells BC of the artificial plant raising site 20A (S20). As the setting of the growing environment, for example, intensity of a light amount of the lighting device 31 is set as a first condition, a level of temperature in the artificial plant raising site 20A is set as a second condition, and the rich and poor of fertilizer to be given to each variant in the artificial plant raising site 20A is set as a third condition. Then, settings having different combinations of the first to third conditions are applied to the growing environmental cells EC.

Next, one kind of variant V4 in which only one gene is genome-edited is prepared (S21). A type of gene to be subjected to genome editing can be freely selected. The type of gene to be subjected to genome editing may be selected on the basis of a result of past cultivation cycle analysis processing. The variant V4 is installed in each of a plurality of growing environmental cells EC in the artificial plant raising site 20A.

Next, while the variant V4 is grown in each growing environmental cell EC, time series data 110 related to a growing environment of each growing environmental cell BC and a growth of the variant V4 is acquired (S22). Specifically, the environmental control device 30 controls each control device in the artificial plant raising site 20A so as to realize the growing environment on the basis of the conditions of the growing environment set in S20. At this time, the environmental information monitor 21 and the growing situation monitor 22 monitor a growth degree, a respiration amount, and the like of each variant in a growing process for each growing environmental cell EC. Then, the generated environmental information data 210 and growing information data 220 are synchronized and accumulated in the information analysis device 10 as the time series data 110,

Next, from the time series data 110 and gene information 111, an analysis model 130 representing a correlation among gene information, a growing environment, and a growth situation is created (S23). Specifically, the analysis model generation unit 120 performs machine learning on a correlation between the growing and growth of the variant V4 from the time series data 110 and the gene information 111 to create the analysis model 130.

Next, a growth simulation is executed using the analysis model 130 (824). In the growth simulation, the simulation execution unit 140 evaluates a difference in growth degree or the like of the variant V4 in different growing environments.

Next, a result of the growth simulation is fed back to genome editing and setting of a growing environment (S25). When processing of 825 is completed, the information analysis device 10 ends the analysis processing corresponding to one cultivation cycle.

The information analysis device 10 can update the analysis model 130 on the basis of the new time series data 110 and gene information 111 obtained by changing the setting of the growing environment and the target of genome editing for each cultivation cycle and collecting the environmental information data 210 and the growing information data 220. In the next cultivation cycle in which feedback has been received from the result of the one-cycle analysis processing described above, it is conceivable to perform cultivation of another edited strain series having different functions and the like in the same growing environment, or to perform cultivation in which the growing environment is changed using the same genome-edited strain series. The analysis model 130 updated by repeating the analysis processing including the feedback can be used for searching for a combination of an optimal genome-edited gone (genome-edited strain) and a growing environment that maximizes the growth degree. That is, a method of using the growth simulation in the information analysis system 1 according to the second embodiment is similar to that of the first embodiment.

### <2-3> Advantageous Effects of Second Embodiment

In the information analysis system 1 according to the second embodiment, one kind of variant in which only one gene is genome-edited is prepared for a target plant to be grown/cultivated, the variant is grown in the closed space under different environmental conditions, and growing data of the variant and environmental data of each environmental condition are acquired in a set. As a result, the information analysis device 10 according to the second embodiment can collect information on the correlation between environment and growth in a certain variant more efficiently than the first embodiment, and can create the analysis model 130 representing the correlation with gene information.

In addition, the information analysis system 1 according to the second embodiment feeds back the result of the growth simulation and performs processing of the next cultivation cycle, so that it is possible to efficiently and accurately search for genetic characteristics optimal for a certain growing environment and target selection of genome editing necessary for obtaining the genetic characteristics, similarly to the first embodiment. As a result, similarly to the first embodiment, the information analysis system 1 according to the second embodiment can realize improvement in productivity of excellent variety plants and creation of vegetation having desired environmental adaptability.

### <3> Third Embodiment

A third embodiment relates to an example of parameters in a production process in which an information analysis system 1 is used. Hereinafter, the information analysis system 1 according to the third embodiment will be described in terms of differences from the first embodiment.

### <3-1> Configuration of Artificial Plant Raising Site 20B

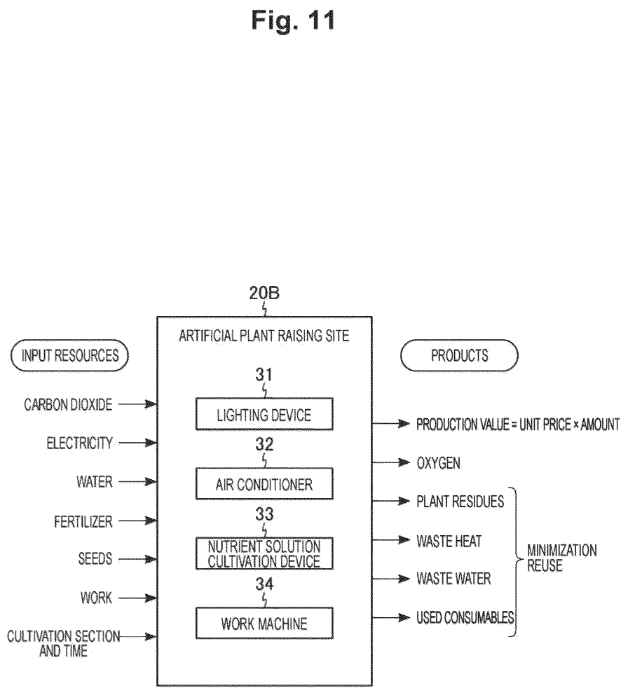

FIG. 11 is a block diagram illustrating an example of a configuration of an artificial plant raising site 20B according to the third embodiment. As illustrated in Fig. 11, the artificial plant raising site 20B includes, for example, a lighting device 31, an air conditioner 32, a nutrient solution cultivation device 33, and a work machine 34. Each of the lighting device 31, the air conditioner 32, the nutrient solution cultivation device 33, and the work machine 34 is controlled by an environmental control device 30. The air conditioner 32 is a control device that controls air conditioning in the artificial plant raising site 208. The nutrient solution cultivation device 33 is a control device which adjusts a supply amount of a nutrient solution to a plant (variant) in the artificial plant raising site 20B. The work machine 34 is a device that manages work related to cultivation in the artificial plant raising site 20B.

### <3-2> Quantification of Parameters in Production Process

Next, an example of quantification of parameters in a production process will be described with reference to Fig. 11. Examples of input resources to the artificial plant raising site 20B include carbon dioxide, electricity, water, fertilizer, seeds, work, a cultivation section, time, and the like. Here, the electricity is used as a power source for the lighting device 31, the air conditioner 32, the nutrient solution cultivation device 33, the work machine 34, and the like in the artificial plant raising site 20B. As the environment of the artificial plant raising site 208, a highly thermally insulated, highly airtight, highly efficient, and clean environment is prepared. Examples of products of the artificial plant raising site 208 include a production value, oxygen, plant residues, waste heat, waste water, and used consumables. Here, the production value is expressed by vegetables or the like obtained as a product, and is expressed by, for example, unit price x production amount. The plant residues, the waste heat, the waste water, and the used consumables correspond to waste generated in production. Other configurations and operations of the configuration of the information analysis system 1 according to the third embodiment are similar to those of the first embodiment.

### <3-3> Advantageous Effects of Third Embodiment>

In order to evaluate production efficiency of a plant factory, it is necessary to quantify input resources and products. For example, in order to obtain the maximum production amount and production value with the minimum input resources and amount of money, it is preferable that the waste is minimum. Also, the waste is preferably reused to the extent possible. Therefore, in the information analysis system 1 according to the third embodiment, as illustrated in FIG. 11, each of the input resources and the product is quantified (quantified).

Then, in the growth simulation, the information analysis system 1 according to the third embodiment searches (predicts) growth conditions under which waste such as plant residuals, used consumables, waste heat, and waste water quantified in the closed space can be minimized or reused, for example. As a result, the information analysis system 1 according to the third embodiment can minimize waste such as waste heat, waste water, and used consumables, and can maximize plant productivity. Note that the third embodiment may be combined with the second embodiment.

### <4> Others

The flowcharts used to describe the analysis processing in the above embodiments are merely examples. In the flowcharts illustrated in FIGS. 6 and 10, the processing order may be changed within a possible range as long as a result similar to that of the embodiment can be obtained, or other processing may be added. For example, the order of 810 and S11 may be interchanged. In the present specification, “synchronizing recording times” may be referred to as “time synchronization”. The information analysis device 10 may be referred to as a “server” or a “processing server”. The CPU 11 may be referred to as a “processor”. Each of the ROM 12, the RAM 13, and the storage device 14 may be referred to as a “storage circuit”. The configurations of the information analysis device 10 and the artificial plant raising site 20 are merely examples. The CPU 11 may be a micro processing unit (MPU), an application specific integrated circuit (ASIC), a field-programmable gate array (FPGA), or the like. The analysis processing may be realized by dedicated hardware. The analysis processing may include both processing executed by software and processing executed by hardware, or may include only one of them. A “connection” only needs to be able to communicate and may be a wired connection, a wireless connection, or a connection via a network.

The present invention is not limited to the above embodiments, and various types of modifications can be made at an implementation stage without departing from the gist of the invention. In addition, the embodiments may be appropriately combined and implemented, and in this case, combined effects can be obtained. Furthermore, the above embodiments include various inventions, and various inventions can be extracted by combinations selected from a plurality of disclosed components. For example, in a case where the problems can be solved and the advantageous effects can be obtained even if some components are deleted from all the components described in the embodiments, a configuration from which the components are deleted can be extracted as an invention.

### Reference Signs List

1 Information analysis system
10 Information analysis device
11 CPU
12 ROM
13 RAM
14 Storage device
15 Communication interface
20, 20A, 20B Artificial plant raising site
21 Environmental information monitor
22 Growing situation monitor
30 Environmental control device
31 Lighting device
32 Air conditioner
33 Nutrient solution cultivation device
34 Work machine
100 Time series data generation unit
110 Time series data
111 Gene information
120 Analysis model generation unit
130 Analysis model
140 Simulation execution unit
210 Environmental information data
220 Growing information data
EC Growing environmental cell
V1, V2, V3, V4 Variant

1. An information analysis method comprising:
setting a growing environment of a plant to be subjected to genome editing in an environmentally controllable closed space;
preparing a plurality of kinds of variants in which one gene of the plant is genome-edited, and growing the plurality of kinds of variants simultaneously in the closed space;
creating time series data in which first data indicating a growing environment of the plurality of kinds of variants and second data indicating a growth state of the plurality of kinds of variants are recorded in time synchronization; and
creating an analysis model representing a correlation among gene information, a growing environment, and a growth state using gene information regarding the plurality of kinds of variants and the time series data.

2. An information analysis method comprising:
growing a variant in which one gene of a plant to be subjected to genome editing is genome-edited in each of a plurality of growing environmental cells in which mutually different growing environments are set, in a closed space including the plurality of growing environmental cells each of which is independently environmentally controlled;
creating time series data in which first data indicating a growing environment of each of the plurality of growing environmental cells and second data indicating a growth state of the variant in each of the plurality of growing environmental cells are recorded in synchronization; and
creating an analysis model representing a correlation among gene information, a growing environment, and a growth state using gene information regarding the variant and the time series data.

3. The information analysis method according to claim 1, wherein
the first data includes any one of a light amount, air temperature, water temperature, a water supply amount, carbon dioxide concentration, nutrients, and time.

4. The information analysis method according to claim 1, wherein
the second data includes any one of a total photosynthesis rate, a dark respiration rate, chlorophyll fluorescence, pore conductance, a leaf area/leaf number distribution, a leaf inclination angle distribution, a root structure and distribution, a chemical component distribution, a net photosynthesis rate, a transpiration rate, a respiration rate, a carbon dioxide application rate, a water supply rate, a water absorption rate, and electric power.

5. The information analysis method according to claim 1, further comprising
simulating growth of the variants using the analysis model, and predicting growing conditions that minimize plant residuals, used consumables, waste heat, and waste water quantified in the closed space.

6. The information analysis method according to claim 1, further comprising
performing machine learning using the time series data as teacher data, and predicting adaptation of the variants when a growing environment changes.

7. An information analyzer that analyzes data obtained by growing a variant in which a gene of a plant is genome-edited in a closed space,
the information analysis device comprising:
a processor configured to execute analysis processing; and
a storage circuit configured to store gene information regarding the variant,
wherein in the analysis processing, the processor further configured to:
record first data indicating a growing environment of the variant and second data indicating a growth state of the variant in time synchronization and creates time series data when receiving the first data and the second data, and store the time series data in the storage circuit; and
create an analysis model representing a correlation among gene information, a growing environment, and a growth state by using the gene information and the time series data.

8. The information analyzer according to claim 7, wherein
in the analysis processing, the processor further configured to perform machine learning using the time series data as teacher data, and predit adaptation of the variant when a growing environment changes.

9. The information analysis method according to claim 2, wherein
the first data includes any one of a light amount, air temperature, water temperature, a water supply amount, carbon dioxide concentration, nutrients, and time.

10. The information analysis method according to claim 2, wherein
the second data includes any one of a total photosynthesis rate, a dark respiration rate, chlorophyll fluorescence, pore conductance, a leaf area/leaf number distribution, a leaf inclination angle distribution, a root structure and distribution, a chemical component distribution, a net photosynthesis rate, a transpiration rate, a respiration rate, a carbon dioxide application rate, a water supply rate, a water absorption rate, and electric power.

11. The information analysis method according to claim 2, further comprising
simulating growth of the variants using the analysis model, and predicting growing conditions that minimize plant residuals, used consumables, waste heat, and waste water quantified in the closed space.

12. The information analysis method according to claim 2, further comprising
performing machine learning using the time series data as teacher data, and predicting adaptation of the variants when a growing environment changes.
